(19) United States
(12) Patent Application Publication
(10) Pub. No.: US 2012/0245482 A1
(43) Pub. Date: Sep. 27, 2012

(54) Anesthesia Monitoring Device and Method

(76) Inventors: Jeffrey W. Bolser, Vadnais Heights, MN (US); Corinne Elizabeth Bolser, Vadnais Heights, MN (US)

(21) Appl. No.: 13/234,622

(22) Filed: Sep. 16, 2011

Related U.S. Application Data
(60) Provisional application No. 61/383,461, filed on Sep. 16, 2010.

(51) Int. Cl. A61B 5/0488; A61B 5/11

(52) U.S. Cl. 600/546

(57) ABSTRACT

A patient monitoring system to measure the depth of anesthesia, and to provide a physician warning during a surgical procedure.

### CROSS REFERENCE TO RELATED CASES

The present application is the utility conversion of U.S. Provisional Patent Application Ser. No. 61/383,461, filed Sep. 16, 2010 entitled Anesthesia Monitoring Device and Method. The provisional application is incorporated by reference in its entirety.

### FIELD OF THE INVENTION

The present invention relates generally to a monitor and companion lead system used in a surgical suite to monitor a patient undergoing a surgical procedure under anesthesia.

### BACKGROUND OF THE INVENTION

In some instances, modern anesthesia employs paralytic agents to prevent tense musculature from interfering with surgical interventions. One method that has been employed to test for muscle paralysis uses a conventional nerve stimulator that applies an electrical signal to skin electrodes and evokes a muscle twitch that is observed by the anesthesiologist. Depending on the location for the surgery, the application of the stimulation may be applied to the radial nerves in the arm while observing the motion of the fingers. Other large and small muscle groups, including the diaphragm and the intercostals muscles, may be observed along with the small muscles of the forehead. For the sake of convenience the forehead is frequently used to observe muscle twitches since it is usually visible to the operator. Large muscle groups of interest to the anesthesia practioner are the intercostals and diaphragm. However depending on the procedure these may not be easily observed due to their proximity to the surgical site.

This method of observing the response to nerve stimulation has many drawbacks. For example, it requires that the anesthesiologist be in a physical position to observe the patient's muscle response; if the anesthesiologist view of the patient's muscle response is blocked due to positioning or sterile draping, the anesthesiologist will not be able to observe and evaluate a patient's response to the stimulation. Further, there is a subjective quality to assessing a patient's response to stimulation; there is no well-defined distinction between a response that is problematic (indicating that additional paralytic agent is required for safe surgery) and a response that is not problematic. Still further, there may be muscle activity that is not visible to the eye that nevertheless indicates that additional paralytics or anesthesia is warranted or will soon be warranted.

### SUMMARY OF THE INVENTION

The present invention includes a monitoring device consol and a companion lead system. The lead system may be applied to the surface of the patient and be under surgical drapes and therefore out of sight of the anesthesiologist. The ability to attach the lead and sensors without regard to visibility allows more flexibility in the positioning of the anesthesiologist in the surgery space and makes it possible to apply an objective standard to identifying problematic muscle movement or activity.

The present invention employs an anesthesia monitoring device that includes accelerometers and EMG patch electrodes to detect both the motion that results from muscle activity as well as the electrical signals associated with muscle activity as well as the electrical signals that actually originate from individual muscles or muscle groups. The preferred part ion of the system has a stand alone monitoring unit coupled to one or more patch electrodes with cables. An accelerometer is incorporated in to the lead system. In general one accelerometer is all that is needed but multiple accelerometer s are envisioned as well. The accelerometers can be used alone or in conjunction with applied nerve stimulation. The accelerometers may be incorporated into electrode patches of various kinds, including patch electrodes and other forms of adhesive skin electrodes. In addition or as another alternative, accelerometers can be incorporated into clip-on structures that grip anatomical features, such as fingers.

Although patch electrodes are slightly preferred, one or more electrode sites may be accessed with minimally invasive fine gauge needle electrodes. Needle electrodes may be useful in some procedures where patch electrodes are difficult to place.

The anesthesia monitor includes electronic components to monitor acceleration transduced by one or more remote accelerometer sensors, as well as electromyography monitoring system (EMG) to detect muscle signals from electrode sites on the patient. The anesthesia monitor device may optionally but preferably include conventional stimulator electronics for delivering “train of four” or other common stimulation patterns to electrodes on the patient. The stimulator portion of the system is coupled to two surface electrodes that supply energy to the musculature. The EMG monitoring portion of the system is connected to two electrodes that will collect electrical potentials associated with muscle motion. The stimulator portion and the EMG monitoring portion may share one electrode. The accelerometer will be mounted on or more likely in the patch that contains an electrode. Multiple accelerometers in multiple sites are contemplated. Therefore in summary the anesthesia monitoring system includes a bedside monitoring unit that has within it a conventional muscle stimulator; an electromyography (EMG) detection system; and a motion detection system. These systems collect data that may be displayed simultaneously on a display screen to a user. The electrodes and accelerometers form a transducer system that may be attached to the surface of a patient. Typically a generally oval patch body is attached to the surface of a patient and it will incorporate at least a first electrode and a second electrode. The accelerometer will be attached to said patch body as well. A cable system will connect the monitoring unit to the transducer system. In general the first electrode is coupled to said muscle stimulator for delivering electrical energy to said transducer system to evoke a muscle twitch and the second electrode is coupled to said electromyography detection system. Since the visual display system presents stimulation data, electromyography data and motion data simultaneously the anesthesiologist may view at the same time muscle motion and relative electrographic activity and inactivity.

### DESCRIPTION OF THE DRAWINGS

Throughout the figures of the drawing, like reference numeral indicate identical structures wherein:

FIG. 1 shows a schematic layout of the anesthesia monitor system;

FIG. 2 shows alternative patch locations;

FIG. 3 shows alternative patch locations;

FIG. 4 shows a patch body and transducer system

FIG. 5 shows a patch body and transducer system;

FIG. 6 shows an integrated multiple patch and transducer system; and,

FIG. 7 shows an anesthesia monitor device with multiple transducer systems.

### DETAILED DESCRIPTION OF THE INVENTION

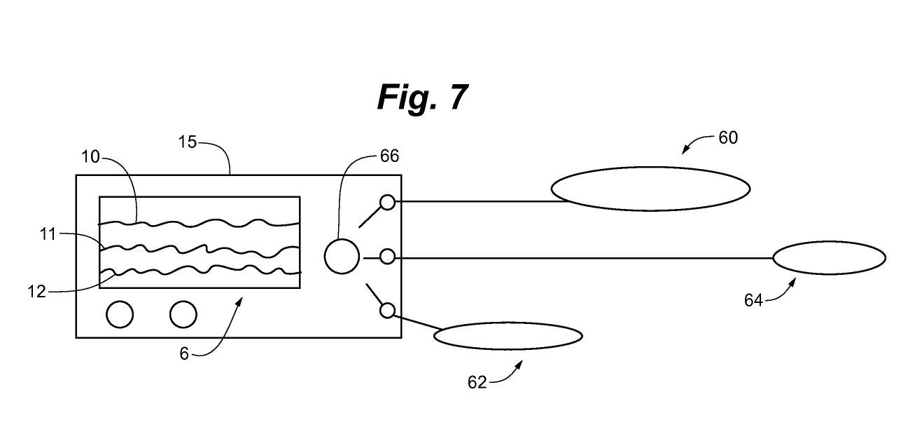

The invention overall is partitioned into one or more patch electrode systems seen at reference numerals 60, 64 and 62 in FIG. 7. The electrodes systems are connected to a monitoring device 15 that incorporates a visual display 6 that presents a tracing 11 reflecting acceleration or motion data taken from a skin mounted accelerometer along with a tracing 10 presenting the real time electromyography sensor data. A further tracing 12 may display the stimulator output. This overall partitioning is seen in FIG. 7 where a user switch 66 may select between patch/sensor systems typified by patch/sensor 60 or 64 or 62. Although multiple patch systems are contemplated the disclosure is clarified by referring to a single patch system embodiment.

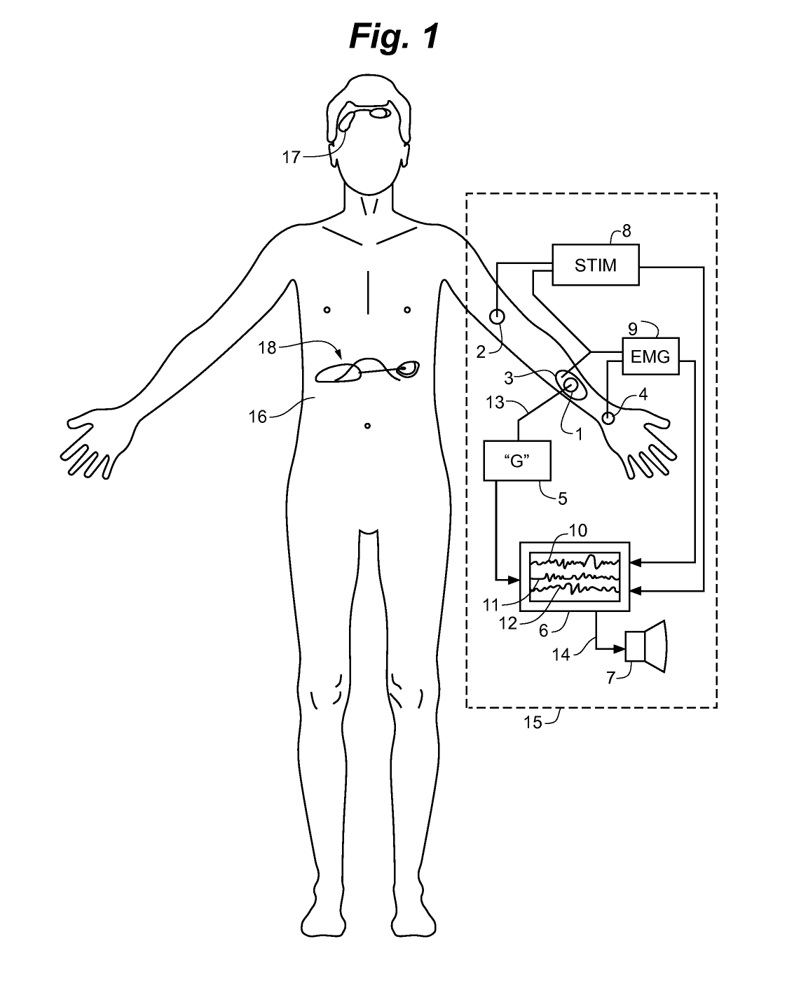

In one embodiment, as depicted in FIG. 1, the anesthesia monitor device of the invention 15 includes one or more electrode patches at reference numeral 2 and reference numeral 3. In this embodiment an accelerometer 1 is incorporated in patch 3 and it is used to detect motion at or near the site of interest, in this instance the forearm of patient 16. The accelerometer 1 is electronically coupled for data communication to a signal receiver and acceleration signal processor 5 which measures the motion and or acceleration of the sensor 1. In this fashion it is in essence a “G” meter. This coupling can be wireless via radio frequency (RF) or it can be wired as shown in the figure with cable 13 connecting the accelerometer 1 with the receiver/processor 5. The signal receiver/processor 5 can be coupled to a visual computer screen type monitor 6 depicting what the accelerometer has sensed, such as by showing a wave form 11 or numerical values or textual description or an excessive motion alarm condition. In addition, or alternatively, the monitor 6 can be coupled by cable 14 to an acoustic alarm 7 that provides a sound or light or other indicator of alarm upon the detection of motion of the patch above some threshold amount. In an alternative embodiment, the processor could be coupled to a device (not shown) controlling the administration of anesthesia or paralytic agents.

Also seen in FIG. 1 is a conventional nerve stimulator 8 that can be used to provide a conventional course of stimulation, for example tentus or train of four stimulation regimes. It is preferred that the stimulator 8 form a component of the overall anesthesia monitoring device 15. As shown in the figure the stimulator 8 may connect to a patch electrode 2 on the body and a patch electrode 3 that has the accelerometer 1 embedded in it or placed upon it. A electromyography “ EMG” measuring device 9 may also connect to a common patch 3 and a reference patch electrode 4 to monitor electrical signals from the muscles. In this embodiment of the method of the invention, the system may also include nerve stimulators that may be incorporated into the accelerometer patches or may be distinct. In use, the patient's nerves are stimulated by an electrical stimulator 8 while the concomitant motion is sensed and/or measured by the attached accelerometers 1 and EMG monitor 9. The sensed motion is quantified and displayed on display monitor 6 and/or compared to a predetermined threshold for an alarm function.

One or more accelerometers 1 can be used in surgical situations in which only anesthesia is used, without paralytics and in surgical situations in which paralytics are employed. In situations with paralytics, the accelerometers can indicate that paralytics are wearing off. In situation without paralytics, the accelerometers can provide useful information about when activity in a muscle indicates it may twitch. When paralytics are not used, muscles will always “twitch” in response to electrical stimulation. Our device in this instance would be to collect impulse signals originating in the target muscles or groups of muscles themselves and alert the operator of an increase in volume or intensity of these signals which probably indicates a deepening of the anesthetic is necessary in order to avoid patient movement during critical surgical manipulation.

The accelerometers employed according to this invention can give objective measures of muscle activity and therefore can eliminate the guesswork or lessen the experience that would otherwise be required to accurately evaluate muscle activity during surgery based on observation alone.

Having an accurate representation of muscle activity during surgery is useful in knowing when intervention is required to deepen anesthesia or provide additional paralytics. This information is useful in preventing the use of more anesthesia or paralytics than is required; using no more of these agents than is required is useful in minimizing adverse side effects.

FIG. 1 also shows two additional monitoring sites. A pair of patches may be applied to the forehead as indicated by reference numeral 17 or a pair of electrodes may be placed across the abdomen at reference numeral location 18 to detect motion of the diaphragm and other muscle groups.

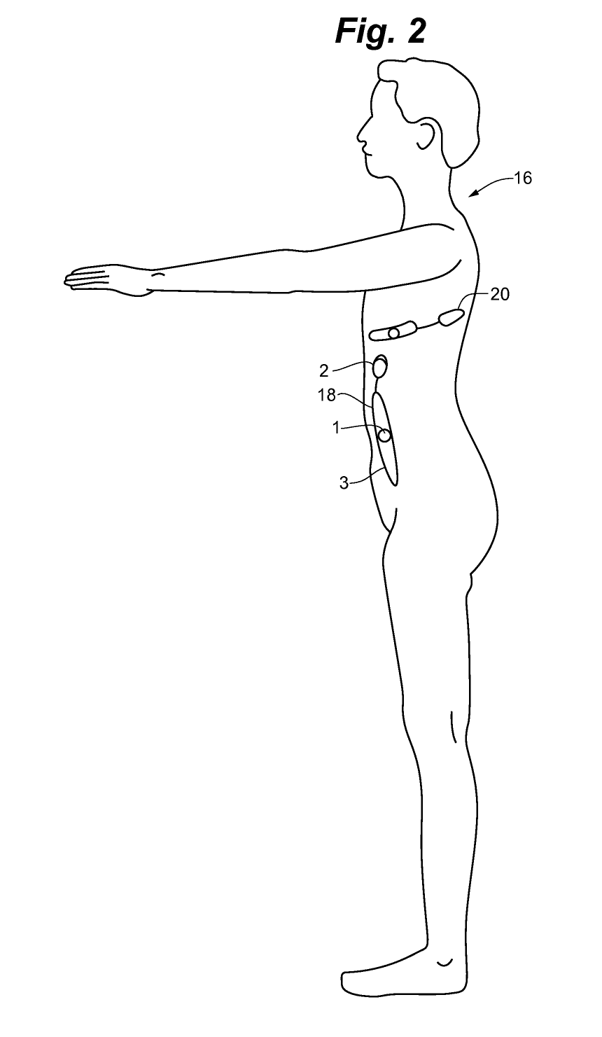

FIG. 2 shows the side of the patient 16 with an electrode system placed on the abdomen with a patch electrode 3 and accelerometer 1 located nearby a reference electrode 2 patch at reference numeral location 18. Another potential electrode location is along the rib cage to monitor the intercostals muscles at reference numeral location 20.

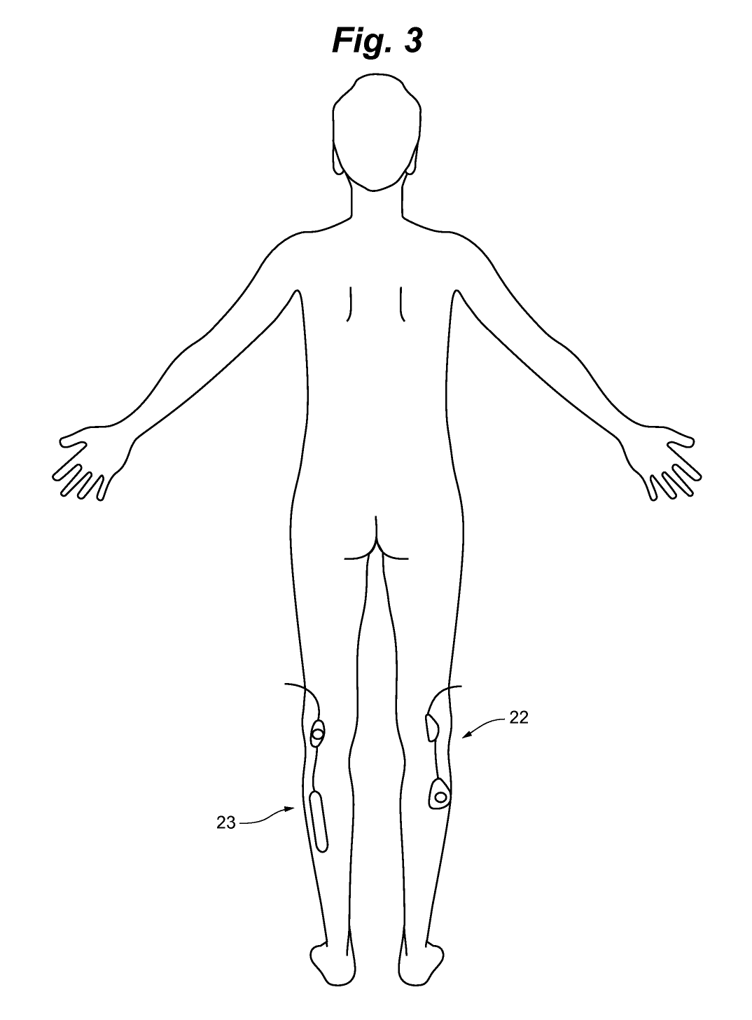

FIG. 3 shows the location of an electrode/accelerometer array on the backs of a patients legs on the left 23 or the right 22. One advantage of the system is that the electrode sites do not need to be directly visible to the operator.

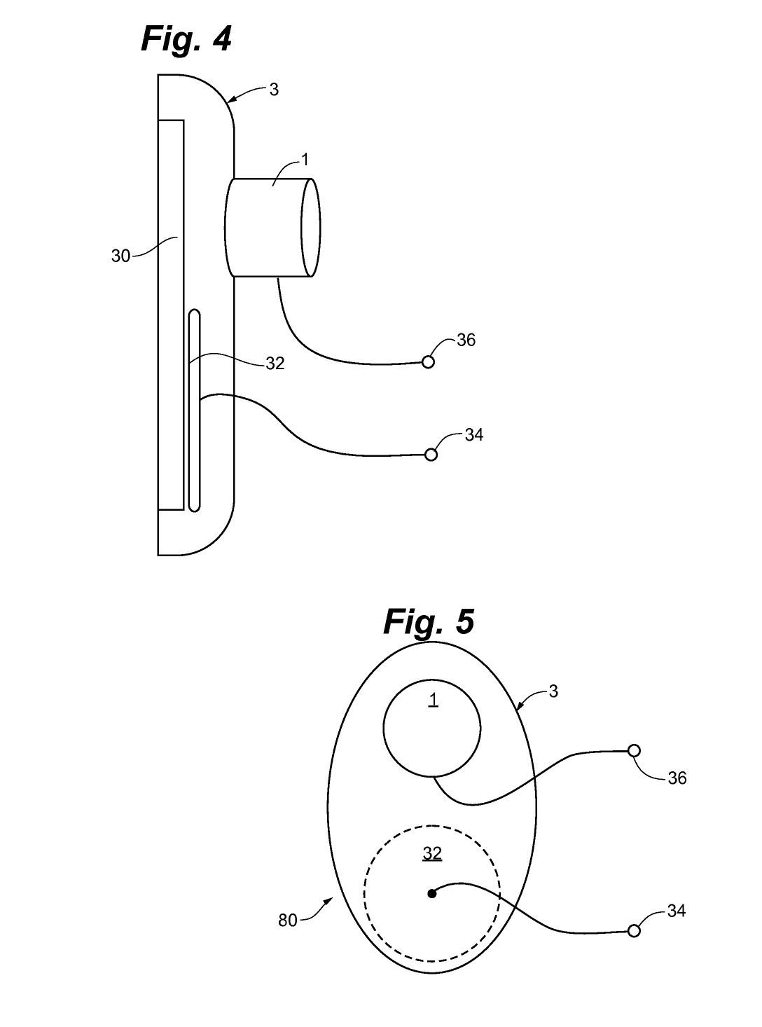

FIG. 4 shows cross section side view of a patch 3 that incorporates an accelerometer 1 mounted on a surface of the patch. an adhesive layer 30 may be used to attach the patch 30 to the patient. Both the electrode 32 and the accelerometer can be coupled to the monitor system via cables 34 and 36.

FIG. 5 shows the patch 3 seen in FIG. 4 in plan view. Here the outline of the patch s generally oval in shape with the accelerometer 1 not overlapping the electrode 32 location.

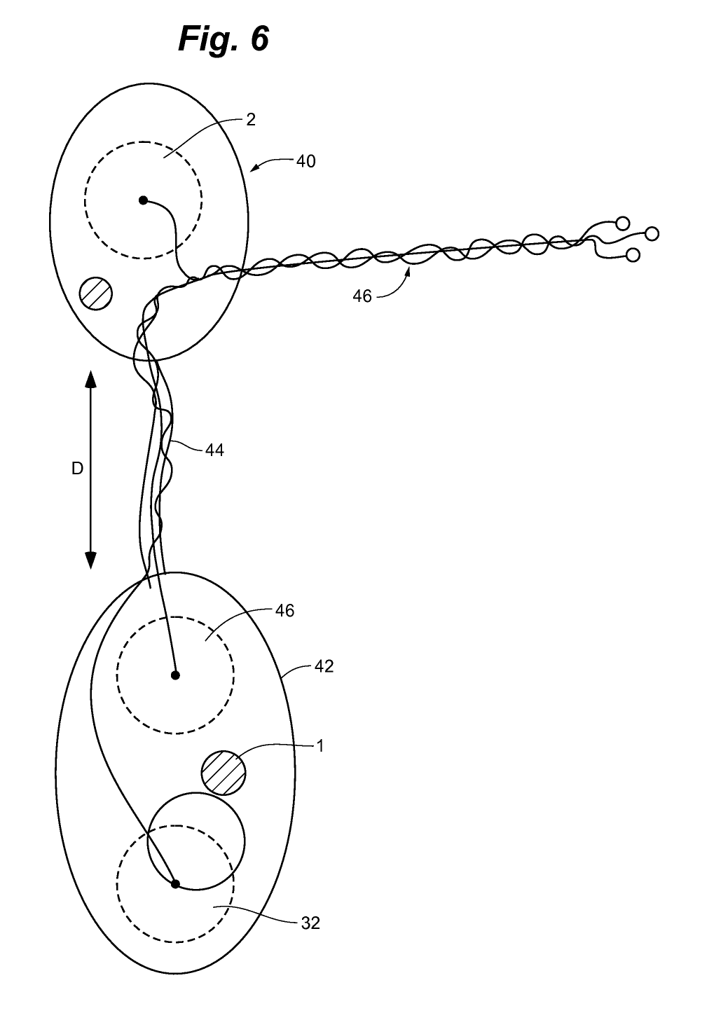

FIG. 6 shows an alternate patch system with two patch elements 40 and 42 connected by a cable 44 that spaces the electrode sites apart. This type of construction can help the operator quickly set up the monitor system. All of the electrical connection to the first electrode 2 , the second electrode 32 and the reference electrode 46 may all be cabled together in a single multithread cable 46 to facilitate connection to the patient.

FIG. 7 shows the use of the monitor 15 with several sets of patches indicated by patch system 60, 62 and 64. A switch 66 can be used to select between patch systems on various parts of the body. This permits the use of multiple sites during a single surgery.

What is claimed is:

1) An anesthesia monitoring system comprising:
a monitoring unit incorporating;
a muscle stimulator;
an electromyography (EMG) detection system;
a motion detection system;
a visual display system;
a transducer system including;
a generally oval patch body attached to the surface of a patient;
a first electrode located in said patch body;
a second electrode located in said patch;
an accelerometer attached to said patch body;
a cable system connecting said monitoring unit to said transducer system;
wherein said first electrode is coupled to said muscle stimulator for delivering electrical energy to said transducer system to evoke a muscle twitch;
wherein said second electrode is coupled to said electromyography detection system;
wherein said visual display system presents Stimulation data, electromyography data and motion data simultaneously thereby permitting analysis of muscle motion and activity and inactivity.
